(19) United States
(12) Patent Application Publication
(10) Pub. No.: US 2011/0020224 A1
(43) Pub. Date: Jan. 27, 2011

(54) APPARATUS AND METHOD FOR PREPARING MEDICINES CONTAINING RADIOACTIVE SUBSTANCES

(75) Inventors: Jacopo Piazzi, Siena (IT); Leonardi Giovannoni, Siena (IT)

Correspondence Address: Pearne & Gordon LLP, 1801 East 9th Street, Suite 1200, Cleveland, OH, 44114-3108, US

(73) Assignee: PHILOGEN S.P.A., 53100 SIENA (IT)

(21) Appl. No.: 12/933,797

(22) PCT Filed: Apr. 16, 2009
(86) PCT No.: PCT/IB2009/051594

§ 371 Date: Sep. 21, 2010

(30) Foreign Application Priority Data
Apr. 17, 2008 (IT) BO2008A000236

(51) Int. Cl. A61K 51/08; A61P 35/00; G21C 1/00

(52) U.S. Cl. 424/1.69; 422/159

(57) ABSTRACT

An apparatus (1) for preparing medicines containing radioactive substances, more specifically injectable medicines containing beta-emitting substances, in which a radioactive element is combined, in a mixture, with a protein to be labelled using the radioactive element; the mixture is then subjected to a chromatographic separation step which separates the mixture and isolates the medicine (16).

### TECHNICAL FIELD

The present invention relates to an apparatus and a method for preparing medicines containing radioactive substances, more specifically injectable medicines containing beta-emitting substances, to which the description below explicitly refers.

As is known, injectable medicines able to emit beta rays are used to eliminate tumour cells in a way that is more selective and less harmful than exposing the whole body to the rays.

The first step of preparation of such medicines involves labelling, or radio-labelling, in which the radioactive substance is incorporated in a protein to create the medicine to be injected.

The whole preparation process must take place in a rigorously sterile and shielded environment. On one hand total sterility of the drug or medicine must be guaranteed, whilst on the other hand protection from harmful exposure to beta rays is required for the operator who supervises the labelling process.

For that reason, in addition to the need to be able to reproduce the medicine with constant quality, manual preparation processes were gradually substituted with automated processes, carried out by suitable apparatuses controlled by an operator.

### BACKGROUND ART

Prior art apparatuses are quite complex and not very practical to manage in the sequence of steps for synthesis of the drug or medicine. Moreover, they are unable to guarantee that the end product is completely cleansed of the by-products of synthesis.

### DISCLOSURE OF THE INVENTION

The present invention has for an aim to provide an apparatus for preparing medicines containing radioactive substances which on one hand can be easily and rapidly used, and on the other hand can produce safe drugs which are free of the by-products of synthesis.

At the same time, the present invention also has for an aim to indicate a method for preparing medicines containing radioactive substances which on one hand can be implemented simply, rapidly and safely by an automatic apparatus, and on the other hand can produce drugs which are safe and free of the by-products of synthesis.

Accordingly, the present invention provides an apparatus for preparing medicines containing radioactive substances comprising the features described in one or more of the appended claims.

Accordingly, the present invention also implements a method for preparing medicines containing radioactive substances comprising the features described in one or more of the appended claims.

### BRIEF DESCRIPTION OF THE DRAWINGS

The invention will now be described, by way of example only and without limiting the scope of the inventive concept, with reference to the accompanying drawings, in which:

FIGS. 1 to 12 are schematic views of a first embodiment of the apparatus according to the present invention, in respective operating steps; and

FIGS. 13 to 16 show respective alternative embodiments of the apparatus illustrated in FIGS. 1 to 12.

### DETAILED DESCRIPTION OF THE PREFERRED EMBODIMENTS OF THE INVENTION

With reference to the accompanying drawings, the numeral 1 denotes as a whole a labelling apparatus for preparing, in a sterile environment, injectable medicines containing radioactive substances, for example but without limiting the scope of the invention, anti-tumour medicines containing beta-emitting substances.

The apparatus 1 comprises a frame 2 on which seven motor-driven valves 3a-3g are mounted, arranged in series, one after another.

The valves 3a-3g are all three-way with quick coupling on their respective actuator 4a-4g.

The actuators 4a-4g, schematically illustrated by respective blocks in the accompanying drawings, are mounted cantilever-style on the frame 2, so that their coupling faces towards the front part of the apparatus 1.

The valve 3a has a first port, communicating with a suction/pumping mouth 5 of a syringe 6, a second port, communicating with a tank 7 containing a washing buffer liquid, and a third port communicating with the first port of the valve 3b. The actuator 4a selectively switches the open/closed state of each of the ports of the valve 3a, controlled by a control unit 23, managed by an operator using a computer 24 connected to the control unit 23.

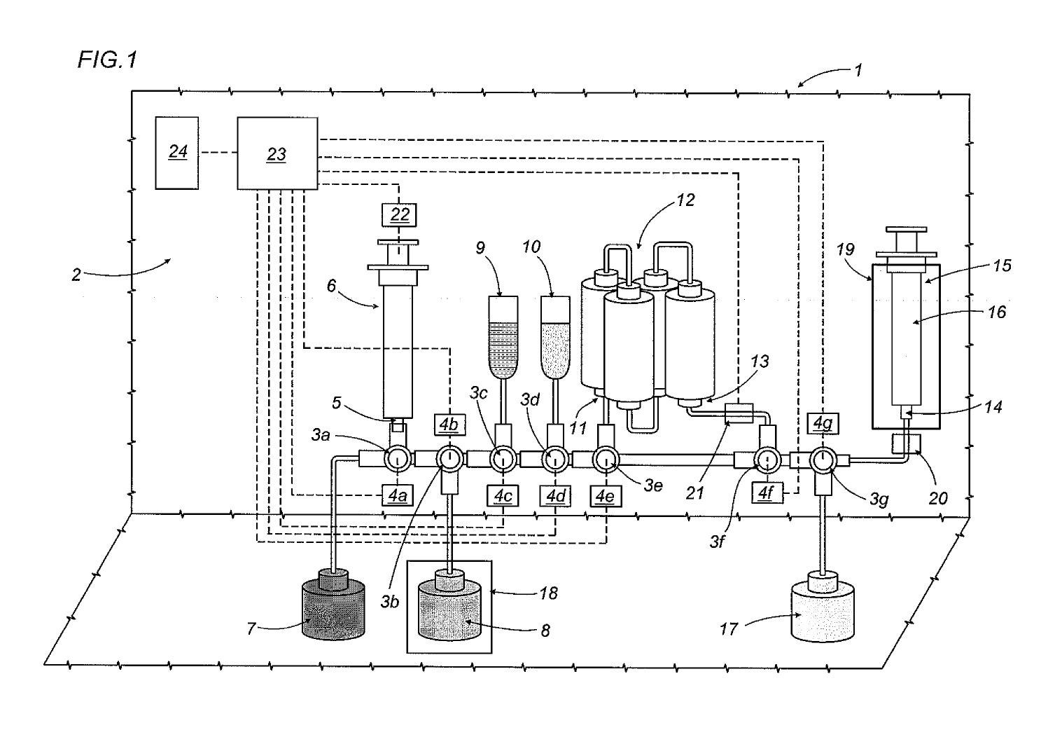

The control unit 23 and the computer 24 are schematically illustrated only in FIG. 1, with respective blocks.

The valve 3b has a second port communicating with a tank 8 containing the beta-emitting radioactive substance, in particular iodine-131, and a third port communicating with a first port of the valve 3c.

Advantageously, iodine-131 is used as the beta-emitting substance because, as well as emitting beta rays, it emits gamma rays, which are particularly useful as a contrast medium for diagnostic scans.

Similarly to the actuator 4a, the actuator 4b selectively switches the open/closed state of each of the ports of the valve 3b, controlled by the control unit 23.

The valve 3c has a second port communicating with a bottle 9 containing a protein to be labelled, for example an antibody, and a third port communicating with a first port of the valve 3d. The actuator 4c also selectively switches the open/closed state of each of the ports of the valve 3c, controlled by the control unit 23.

The valve 3d has a second port communicating with a bottle 10 containing a reagent, specifically chloramine-T, and a third port communicating with a first port of the valve 3e. The actuator 4d also selectively switches the open/closed state of each of the ports of the valve 3d, controlled by the control unit 23.

The valve 3e has a second port communicating with an inlet 11 of a chromatography column 12, and a third port communicating with a first port of the valve 3f.

The chromatography column 12 is a chromatographic separation means.

The suction and pumping syringe 6, the tank 8 containing the radioactive element, the bottles 9 and 10 together with the elements of the apparatus 1 located downstream of them, contribute to forming a circuit in which the substances of which the medicine will be composed and/or used in its preparation pass.

The actuator 4e also selectively switches the open/closed state of each of the ports of the valve 3e, controlled by the control unit 23.

The valve 3f has a second port communicating with an outlet 13 of the chromatography column 12, and a third port communicating with a first port of the valve 3g. The actuator 4f also selectively switches the open/closed state of each of the ports of the valve 3f, controlled by the control unit 23.

Advantageously, along the pipe connecting the outlet 13 to the valve 3f there is a sensor 21 for detecting the composition of the fluid in transit. The sensor 21 is connected to the control unit 23.

The valve 3g has a second port communicating, through a filter 20, with a loading mouth 14 of a disposable syringe 15 for containing the medicine produced 16, and a third port communicating with a tank 17 for collecting the by-products of synthesis.

Similarly to the actuators 4a-4f, the actuator 4g also selectively switches the open/closed state of each of the ports of the valve 3g, controlled by the control unit 23.

Both the tank 8, containing the iodine-131, and the syringe 15, containing the medicine 16 in which the iodine-131 was incorporated, are contained in respective lead shielding jackets 18, 19.

Of the apparatus 1, the tanks 7, 8 and 17, the bottles 9 and 10, the valves 3a-3g, the chromatography column 12, the filter 20, the syringes 6 and 15, with the exception of the actuator 22 which operates the piston, are suitably of the disposable type.

Like the actuators 4a-4g, the actuator 22 is operated under the control of the control unit 23.

The following is a description of apparatus 1 operation starting with the configuration illustrated in FIG. 1 in which the communicating route between the syringe 6 and the tank 7 is open, whilst the communicating route between the valves 3a and 3b is closed. Moreover, in that starting configuration the communicating routes between the adjacent valves are open (with the exception of that between valves 3e and 3f), as are the routes for communicating with the tank 17 and with the inlet 11 and outlet 13 of the chromatography column 12. Closed communicating routes are those with the bottles 9 and 10 and the tank 8 and the syringe 15. The production cycle for a disposable sterile syringe 15 begins with washing of the circuit interposed between the valve 3a upstream and the valve 3g downstream, and in particular of the chromatography column 12.

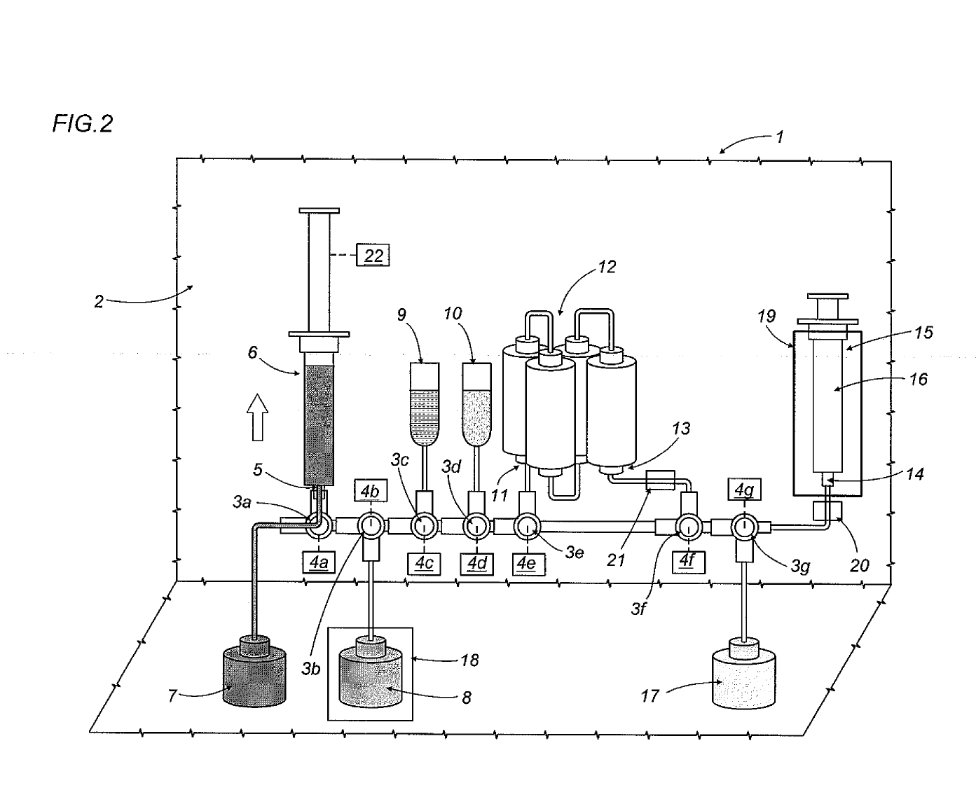

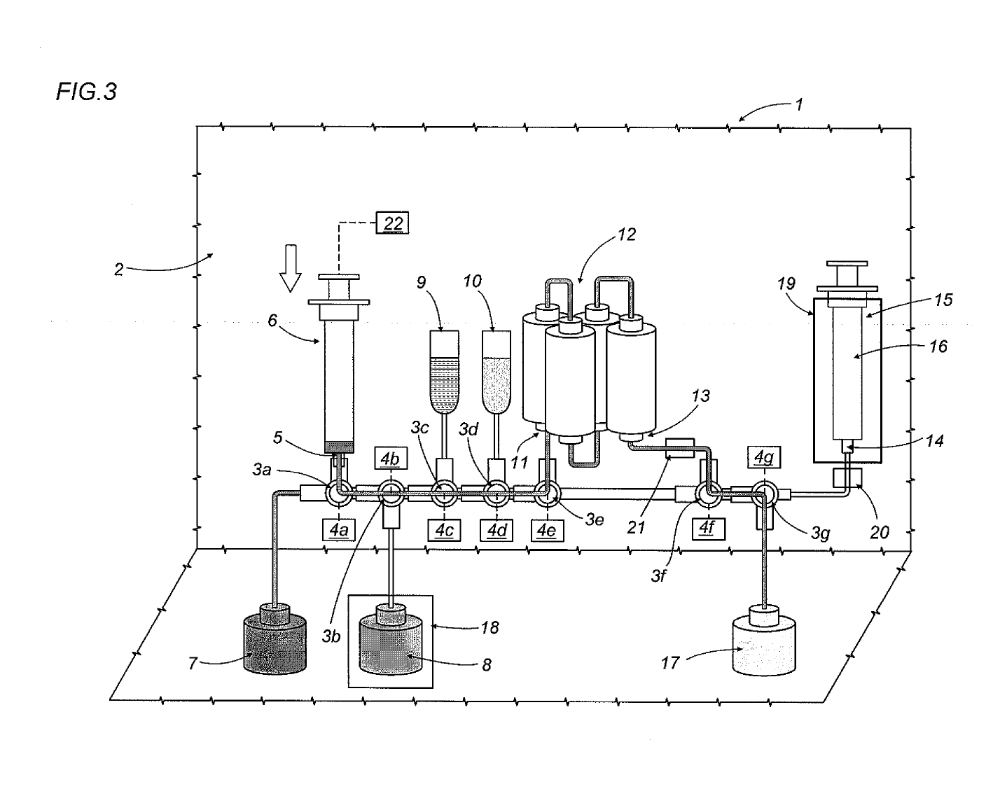

The actuator 22 operates the syringe 6 to draw the washing buffer liquid from the tank 7 (FIG. 2) and to inject it into the circuit interposed between the valve 3a upstream and the valve 3g downstream (FIG. 3).

During the step of drawing the buffer liquid from the tank 7, the valve 3a puts the syringe 6 in communication with the tank 7, keeping the port for communicating with the valve 3b closed.

During the following step of injecting the buffer fluid into the above-mentioned circuit, the communicating route between the valve 3a and the valve 3b is opened, whilst that with the tank 7 is closed. At the same time, the routes for communicating with the bottles 9 and 10, with the tank 8 and with the syringe 15 are kept closed, so that the buffer fluid can pass through all of the valves and the chromatography column 12 before being drained into the tank 17.

In the example illustrated, the chromatography column 12 comprises four elements in series, each having a capacity of 30 cl, and the syringe 6 has a capacity of 50 cl. The syringe 6 is operated at least three times one after another to inject at least 150 cl of buffer liquid into the above-mentioned circuit.

Obviously, the chromatography column 12 may comprise elements in series whose number is different to four, and/or which have a different capacity.

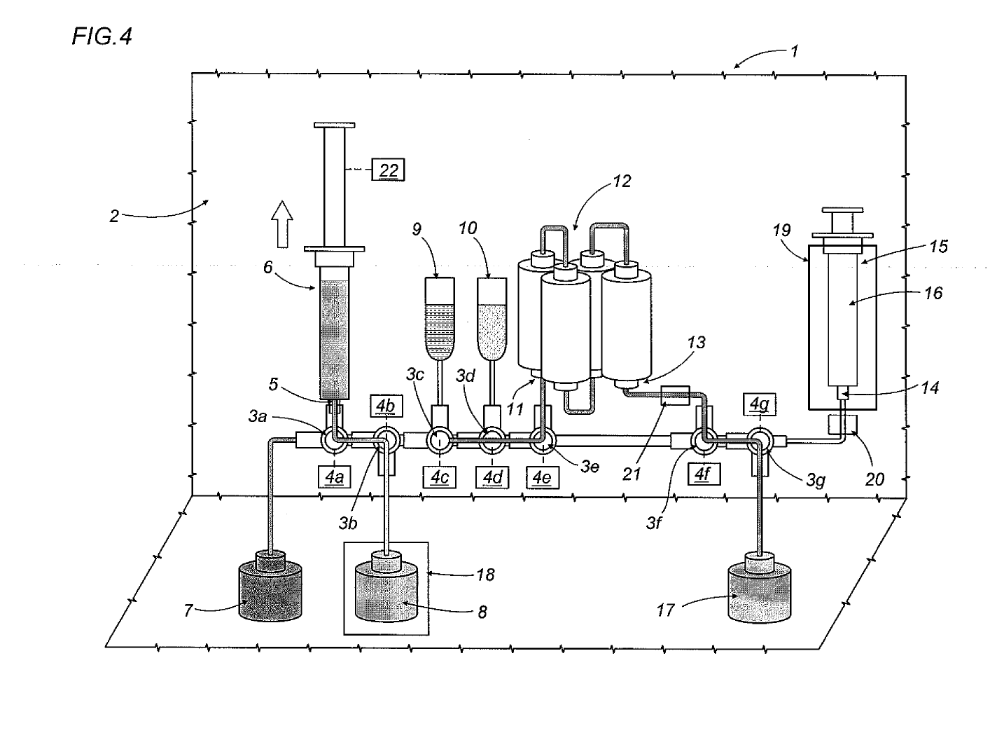

The subsequent step, illustrated in FIG. 4, involves sucking the iodine-131 out of the tank 8. This step is carried out by closing the communicating route between the valves 3b and 3c and putting the syringe 6 in communication with the container 8.

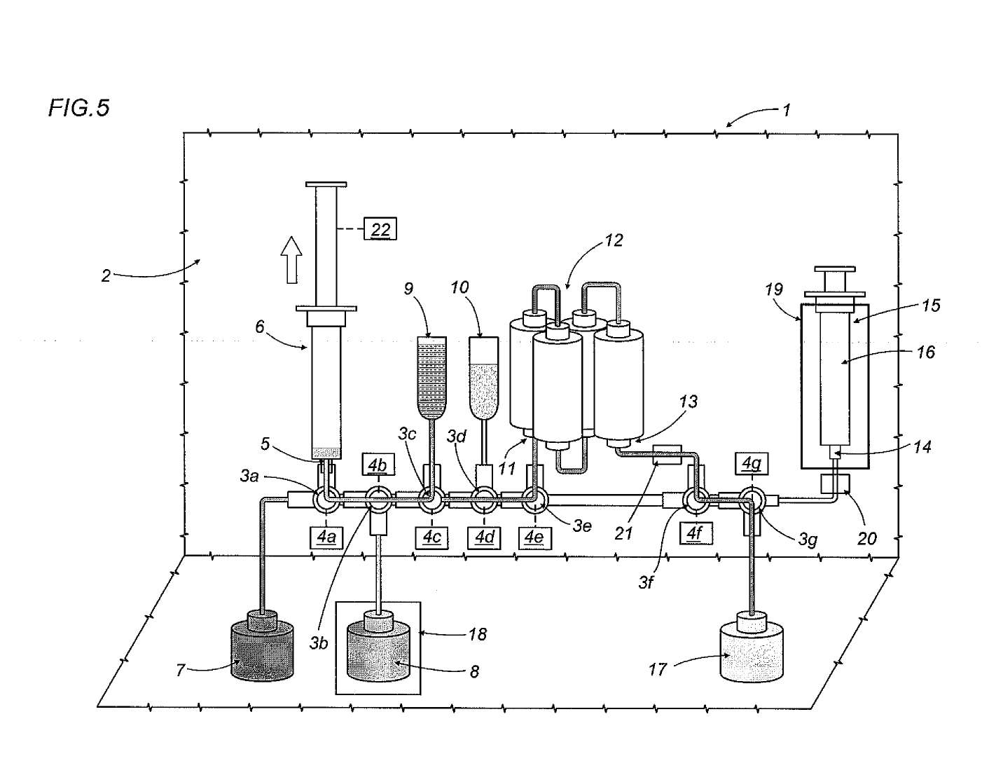

Then, the iodine-131 is mixed with the protein in the bottle 9. This step, illustrated in FIG. 5, is carried out by bringing the syringe 6 from the suction step to the pumping step, after closing the communicating route between the syringe 6 and the container 8, and after putting the syringe 6 in communication with the bottle 9, with the communicating route between the valves 3c and 3d closed.

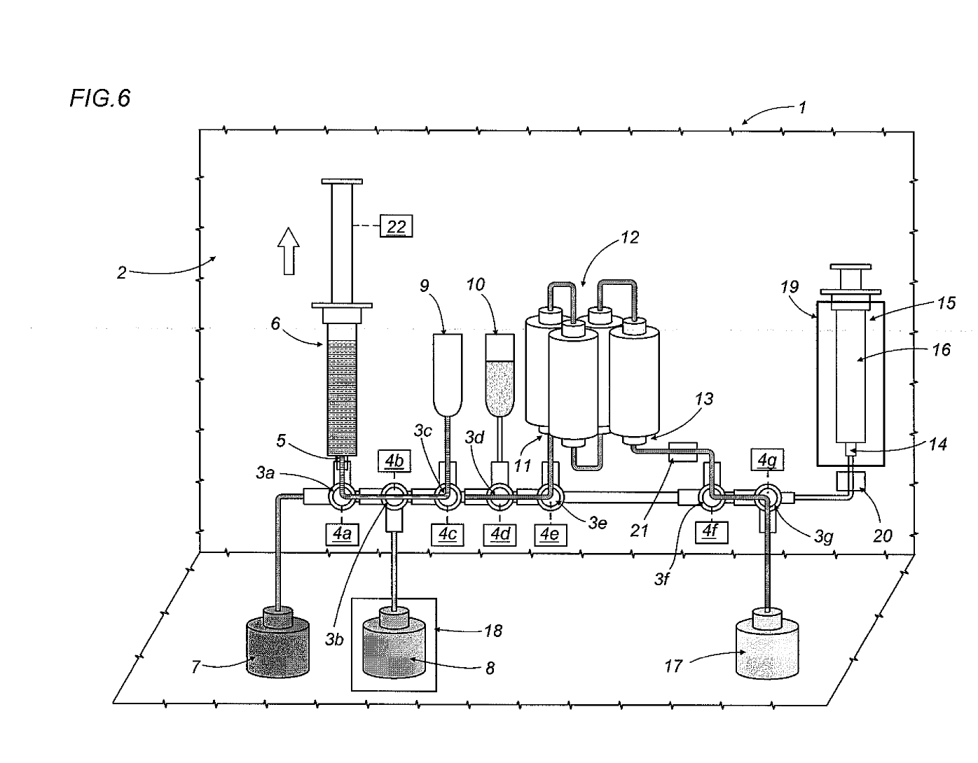

Then, keeping the state of the valves 3a-3g unchanged, the contents of the bottle 9 are sucked out by the syringe 6 (FIG. 6).

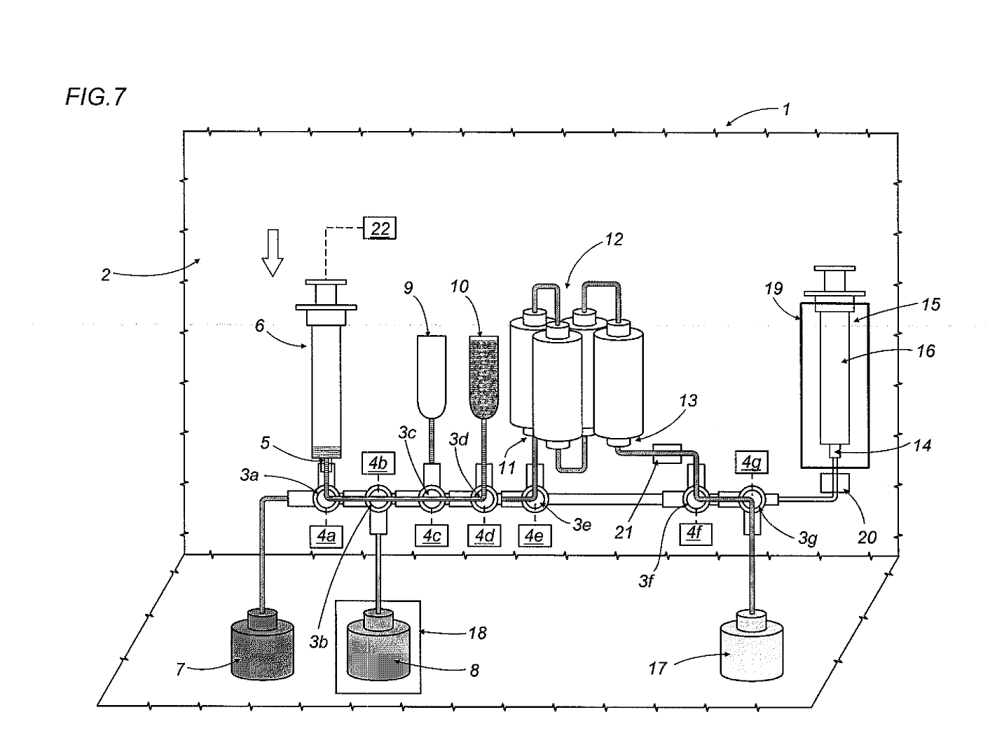

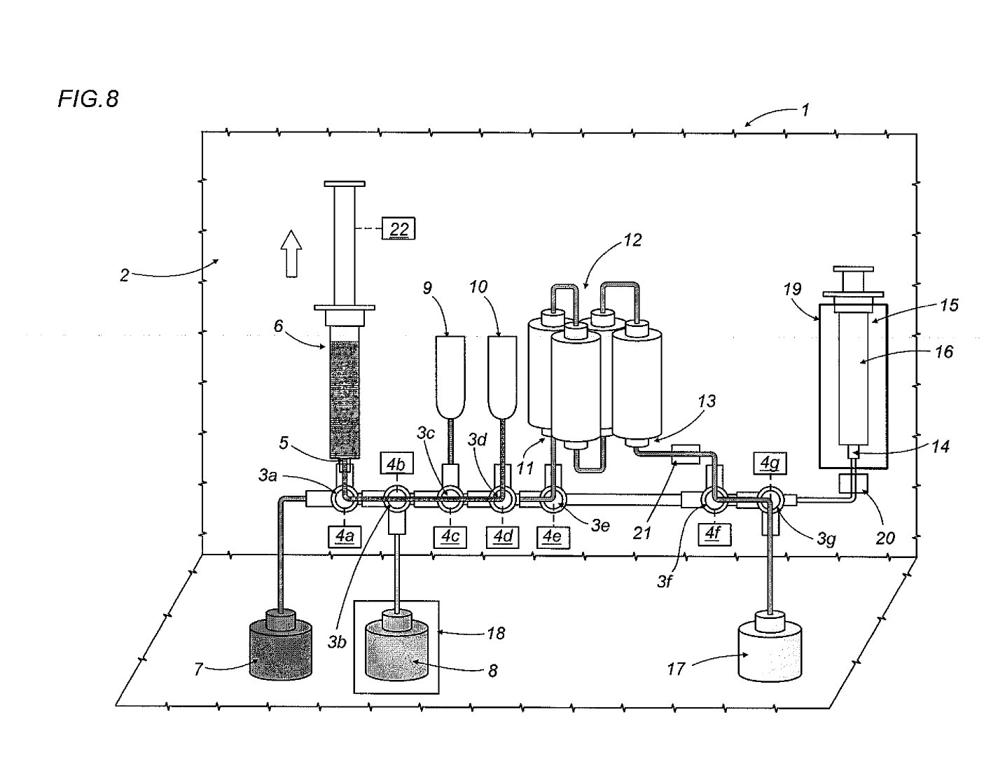

At this point, as illustrated in FIG. 7, the contents of the syringe 6 are poured into the bottle 10, after closing the route for communicating with the bottle 9 and after opening the route for communicating with the bottle 10, with the communicating route between the valves 3d and 3e closed. During this step, the chloramine-T oxidises the protein, which therefore reacts with the iodine-131, incorporating it. In order for the oxidation and subsequent incorporation processes to be completed correctly, this step must go on for at least three minutes. During that period of time, the syringe 6 performs several suction and pumping cycles from and to the bottle 10 (FIG. 8), thus mixing well and homogenising the mixture consisting of the protein, the chloramine-T and the iodine-131.

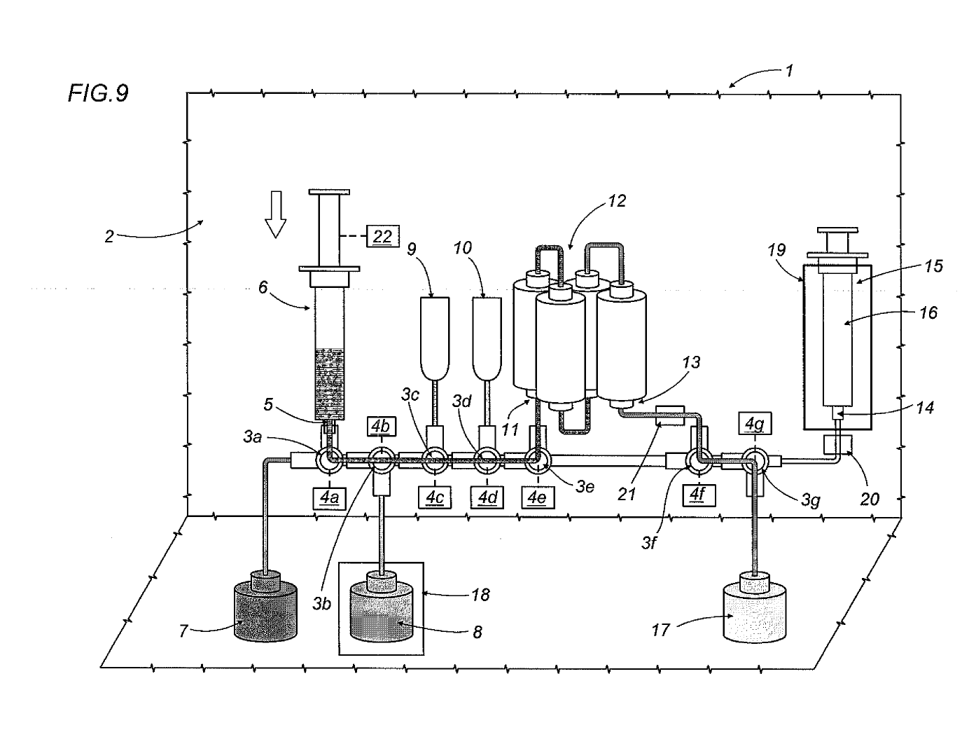

Upon completion of mixing of the protein with the reagent chloramine-T and with the iodine-131, the mixture obtained in this way is sent to the chromatography column 12, where the medicine is separated from the by-products of synthesis. As shown in FIG. 9, the contents of the syringe 6 are poured into the chromatography column 12, after closing the route for communicating with the bottle 10 and after opening the communicating route between the valves 3d and 3e.

Advantageously, the sensor 21 continuously detects the properties of the fluid which, after the chromatographic separation process, comes out of the column 12, in particular detecting the instantaneous quantity of protein in transit, the activity, or the instantaneous quantity of radioactivity in transit, and the conductivity, or the instantaneous salt concentration of the fluid in transit.

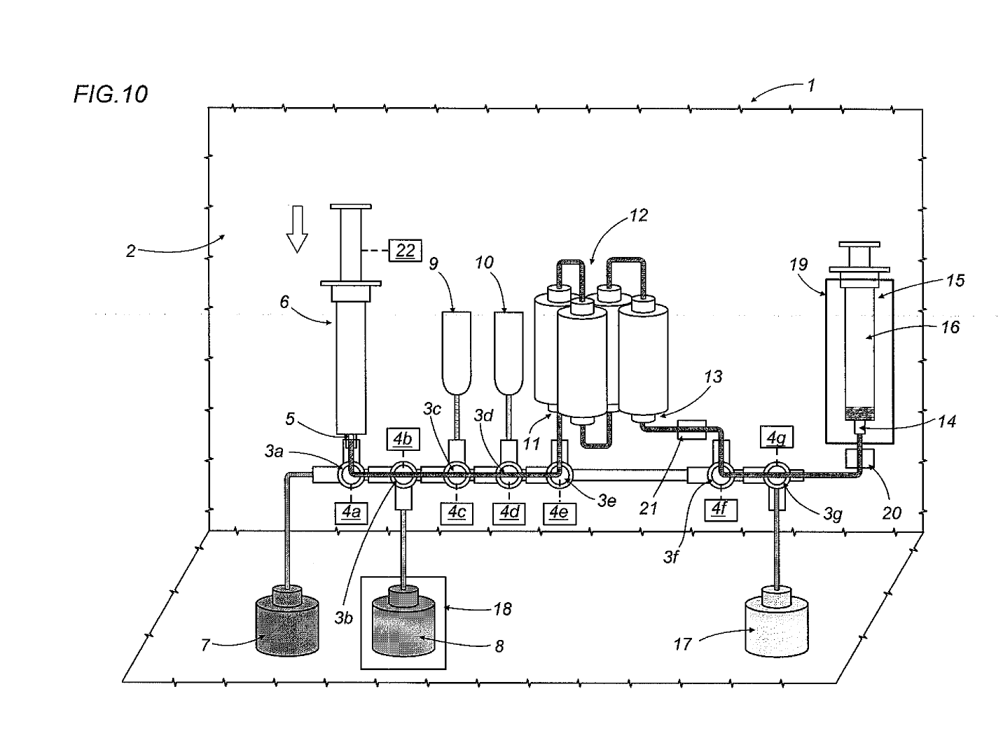

Based on the signals supplied by the sensor 21, or, according to an alternative embodiment, when a known period of time of the chromatographic separation process has elapsed, when one can be sure that the fluid in transit is the desired medicine, perfectly cleansed of the by-products of synthesis, in particular of the excess iodine-131 which did not react and chloramine-T, the route for communicating with the tank 17 is closed and the route for communicating with an acceptance path leading to the syringe 15 is opened (FIG. 10).

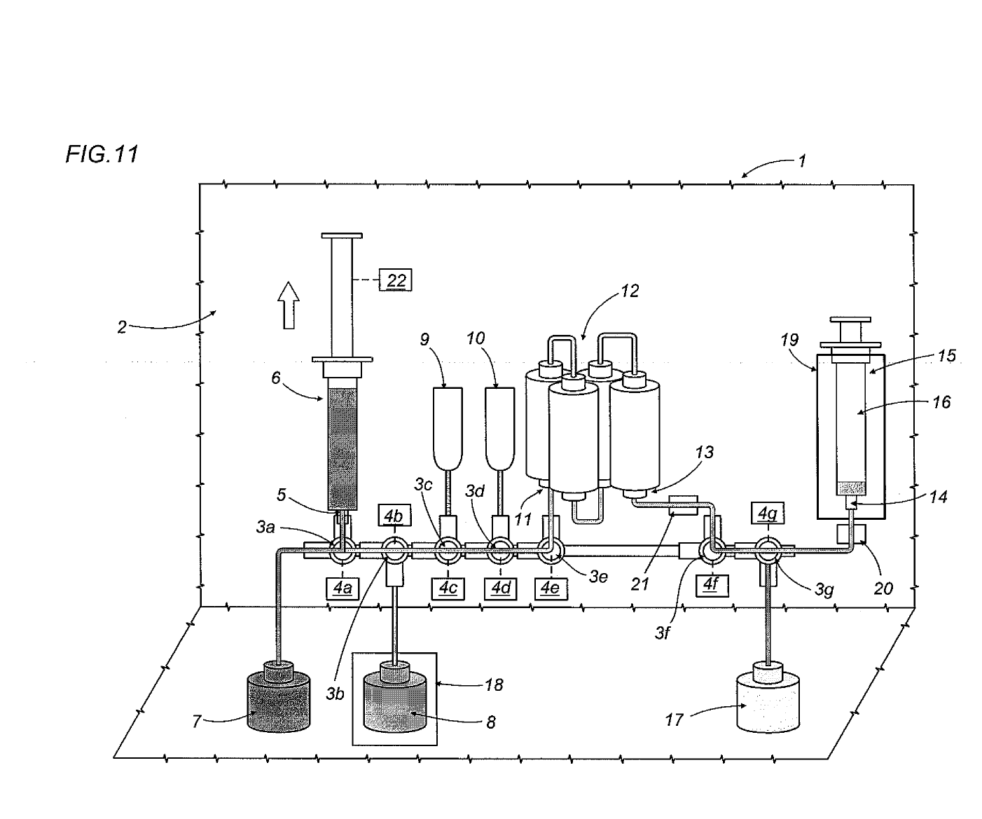

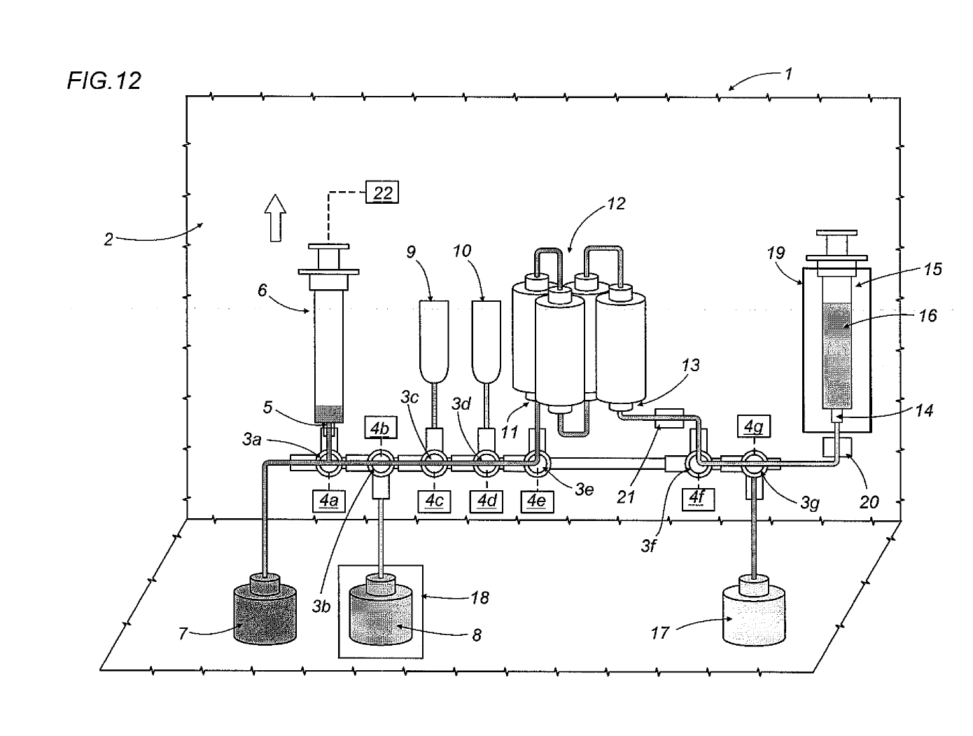

To introduce the medicine contained in the chromatography column 12 and in part of the above-mentioned circuit into the syringe 15, the medicine is pushed upstream by the buffer solution, sucked out of the tank 7 then pumped into the chromatography column 12 by the syringe 6 (FIGS. 11 and 12).

Based on the signals supplied by the sensor 21, or, according to an alternative embodiment which does not comprise the sensor 21, after a known period of time which depends on the capacity of the circuit, when one can be sure that the quality level of the medicine in transit has reached a preset minimum threshold, the route for communicating with the syringe 15 is closed and the route for communicating with the tank 17 is re-opened.

Advantageously, the above-mentioned disposable component parts of the apparatus 1 are grouped together and supplied in a kit so that a new kit is installed for each new medicine preparation operation and, at the end of said preparation, the kit is removed and disposed of.

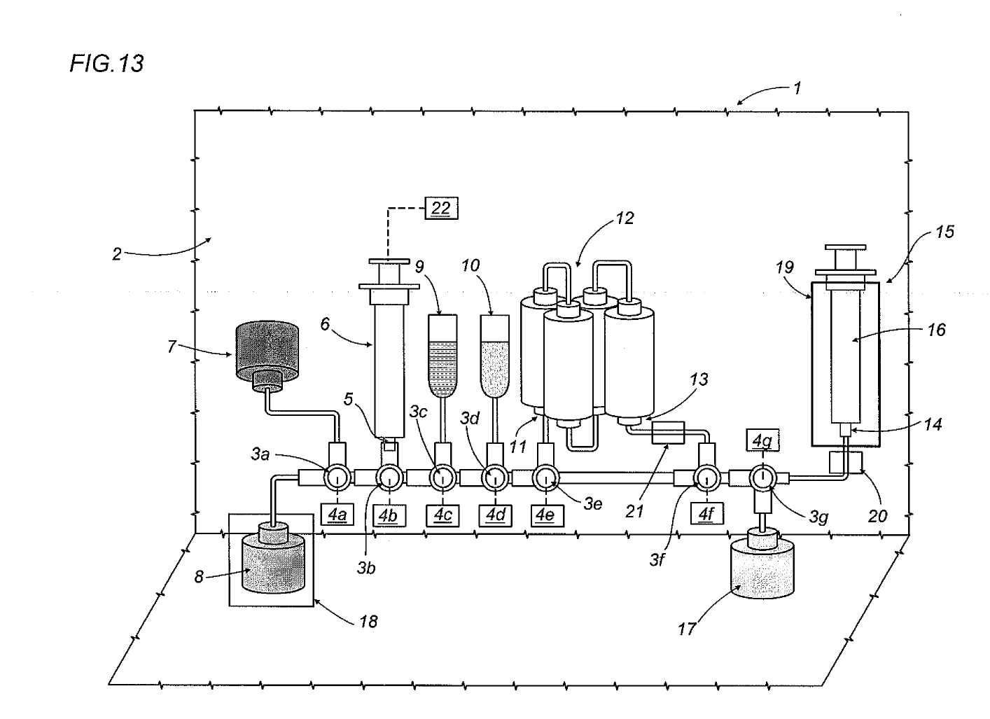

According to the alternative embodiment in FIG. 13, the tank 8 is directly connected to the valve 3a instead of the valve 3b.

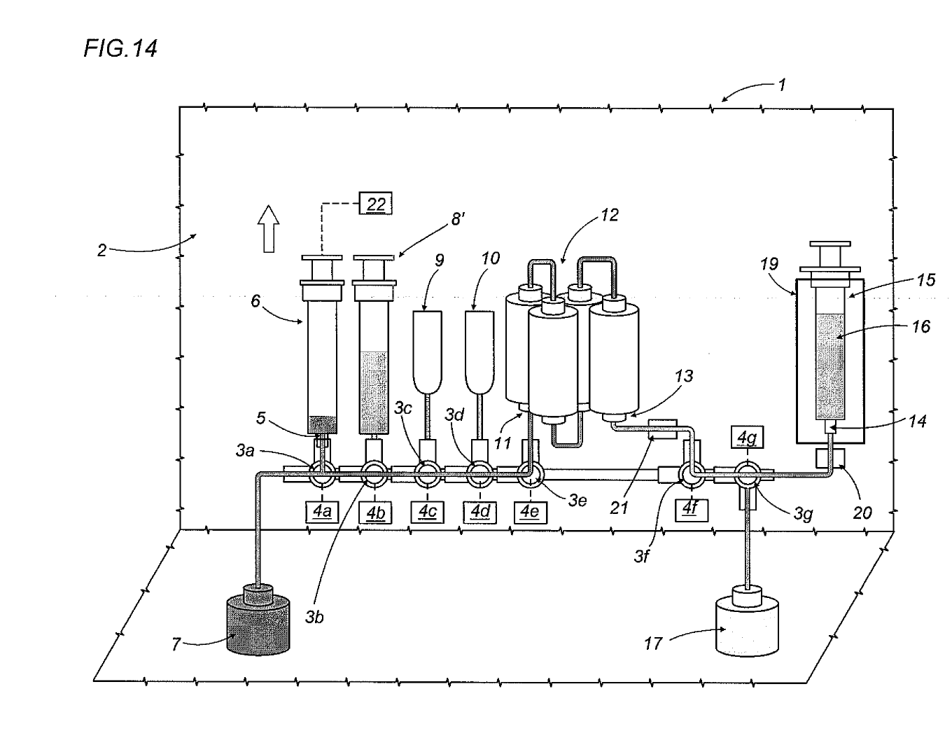

According to the alternative embodiment illustrated in FIG. 14, in place of the bottle 8, the container for the radioactive element is a syringe 8′ with a respective lead shielding jacket 18′.

Advantageously, use of the syringe 8′ allows more practical and safer handling of the container for the radioactive element.

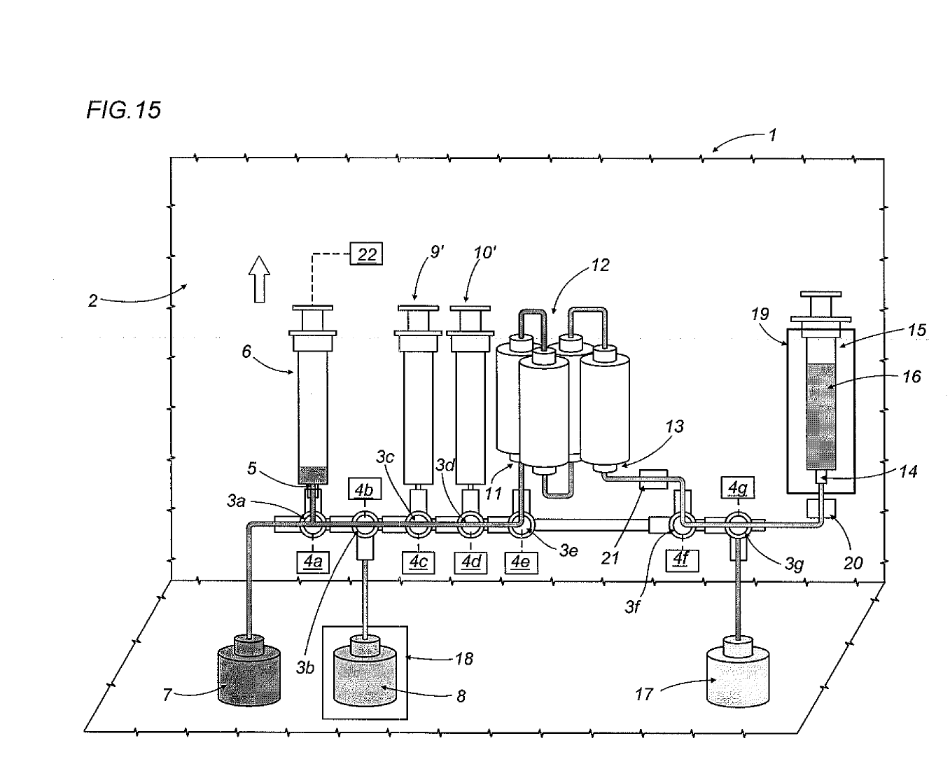

According to the alternative embodiment illustrated in FIG. 15, in place of the bottles in 9 and 10, the containers for the protein and the chloramine-T are respective syringes 9′, 10′.

Advantageously, the use of the syringes 9′, 10′ allows rapid handling of the respective products contained in them.

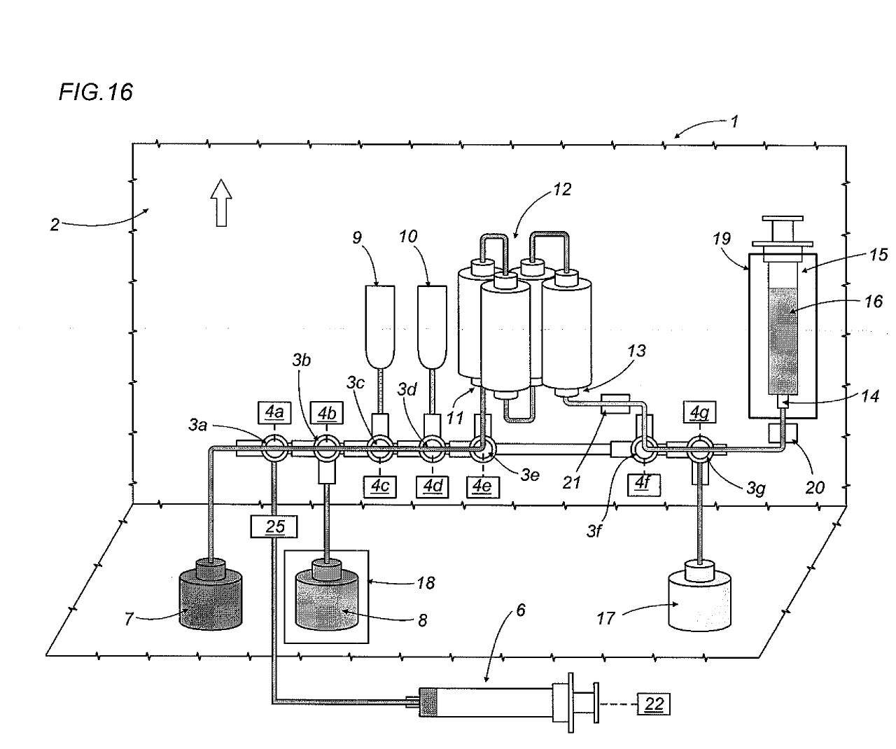

According to the additional alternative embodiment illustrated in FIG. 16, the syringe 6 is connected to the mouth of the valve 3a with a pipe interposed between them.

Along the pipe there is a pressure sensor 25, designed to measure the pressure of the fluid in the pipe from one moment to the next.

Advantageously, the instantaneous pressure measurement carried out by the sensor 25 allows any pressure changes in the pipe to be detected. Such pressure changes indicate malfunctions and/or blockages in the pipe or in other parts of the apparatus located downstream of the pipe and in fluid communication with it.

The invention described, designed in this way, brings important advantages. Given its compact dimensions, the apparatus according to the present invention may be inserted in isolators or shielded hoods already present in hospital departments dedicated to radiopharmaceuticals, guaranteeing maximum protection from radiation for personnel responsible for the production process for radiopharmaceuticals or medicines compared with what is currently the case with manual production procedures during which operators must put their arms in the isolator, meaning that they are not isolated from radioactive emissions.

Additional advantages are offered by the use of disposable components for the apparatus described above, these advantages comprising:
the fact that the substances of which the drug will be composed only make contact with sterile elements;
the fact that there is no need for procedures for sterilising the system flow lines before the start of the process, since the disposable components are advantageously supplied sterile;
the fact that there is no need for procedures for cleaning the system at the end of the process, since all of the components of the flow line are removed and disposed of;
the extremely simple assembly of the flow line component parts;
the elimination of any problems deriving from wear on parts in contact with the drug, since a new disposable kit is used for each new labelling operation.

The invention described above is susceptible of industrial application and may be modified and adapted in several ways without thereby departing from the scope of the inventive concept. Moreover, all details of the invention may be substituted by technically equivalent elements.

1. An apparatus for preparing medicines containing radioactive substances, comprising means for preparing a mixture containing a radioactive element and a protein to be labelled with the radioactive element; the apparatus (1) being characterised in that it comprises chromatographic separation means (12) connected downstream of the preparation means for separating and isolating the medicine (16) from the mixture.

2. The apparatus according to claim 1, characterised in that the preparation means comprise a circuit connected to at least a suction and pumping syringe (6), a tank (8) for containing the radioactive element, a bottle (9) for containing the protein to be labelled and a bottle (10) for containing a reagent.

3. The apparatus according to claim 1, characterised in that it comprises means (6, 7) for washing the chromatographic separation means (12).

4. The apparatus according to claim 1, characterised in that it comprises means (21) for detecting the properties of the fluid which, after the chromatographic separation process, comes out of the chromatographic separation means (12).

5. The apparatus according to claim 4, characterised in that the detecting means (21) are designed to detect the instantaneous quantity of protein in transit, the instantaneous quantity of radioactivity in transit and the instantaneous salt concentration of the fluid in transit.

6. The apparatus according to claim 1, characterised in that it comprises means (25) for detecting the pressure inside the system.

7. The apparatus according to claim 1, characterised in that it comprises a tank (17) for collecting the by-products of synthesis and a controlled valve (3g), interposed between the chromatographic separation means (12) and the collecting tank (17) for sending the fluid coming out of the chromatographic separation means (12) to the collecting tank (17) or to a medicine (16) acceptance path.

8. The apparatus according to claim 7, characterised in that it comprises a control unit (23) for controlling the valve (3g).

9. The apparatus according to claim 4, characterized in that it comprises a control unit (23) for controlling the valve (3q), and further characterised in that the control unit (23) is connected to the detecting means (21) so that it can control the valve (3g) on the basis of the information received from the detecting means (21).

10. The apparatus according to claim 8, characterised in that the control unit (23) controls the valve (3g) on a predetermined time basis.

11. The apparatus according to claim 1, characterised in that it is at least partly disposable.

12. A method for preparing medicines containing radioactive substances in an apparatus according to claim 1, comprising the step of combining, in a mixture, a radioactive element with a protein to be labelled using the radioactive element; the method being characterised in that it comprises a chromatographic separation step by means of which the mixture is separated and the medicine (16) is isolated.

13. The method according to claim 12, characterised in that in addition to the radioactive element, in the mixture a reagent is also combined with the protein to be labelled.

14. The method according to claim 13, characterised in that the reagent is combined with the protein after the radioactive element.

15. The method according to claim 13, characterised in that the reagent is an oxidiser for the protein.

16. The method according to claim 12, in which the chromatographic separation step is carried out using a separation column (12), characterised in that it comprises a preliminary step of washing the column (12).

17. The method according to claim 16, characterised in that the washing step is carried out by making a buffer solution pass through the column (12).

18. The method according to claim 12, in which the chromatographic separation step is carried out using a separation column (12), characterised in that it comprises a step of detecting the properties of the fluid which, after the chromatographic separation process, comes out of the column (12).

19. The method according to claim 18, characterised in that the detecting step is continuous.

20. The method according to claim 19, characterised in that the detecting step involves detection of the instantaneous quantity of protein in transit, the instantaneous quantity of radioactivity in transit and the instantaneous salt concentration of the fluid in transit.

21. The method according to claim 18, characterised in that the fluid coming out of the column (12) is sent to a tank (17) for collecting the by-products of synthesis or to a medicine (16) acceptance path, depending on the result of the detecting step.

22. The method according to claim 12, in which the chromatographic separation step is carried out using a separation column (12), characterised in that the fluid coming out of the column (12) is sent to a tank (17) for collecting the by-products of synthesis or to a medicine (16) acceptance path according to a predetermined time basis.

23. The method according to claim 12, wherein the radioactive element is iodine-131.

24. The method according to claim 12, wherein in addition to the radioactive element, in the mixture a reagent consisting of chloramine-T is also combined with the protein to be labelled.

25. The method according to claim 12, characterised in that the chromatographic separation step is preceded by a step of mixing the mixture well.

26. The method according to claim 25, characterised in that the step of mixing the mixture well goes on for at least three minutes.
